(19) United States
(12) Patent Application Publication
(10) Pub. No.: US 2012/0294813 A1
(43) Pub. Date: Nov. 22, 2012

(54) Functional branched polyether copolymers and method for the production thereof

(76) Inventors: Holger Frey, Emmendingen (DE); Daniel Wilms, Mainz (DE); Frederik Wurm, Wiesbaden (DE)

(21) Appl. No.: 13/516,030

(22) PCT Filed: Dec. 10, 2010
(86) PCT No.: PCT/EP2010/007553

§ 371 Date: Jun. 14, 2012

(30) Foreign Application Priority Data
Dec. 18, 2009 (DE) 10 2009 059 104.4

(51) Int. Cl. C07C 41/03; A61K 8/86; A61Q 17/04; C07C 43/13

(52) U.S. Cl. 424/59; 568/620; 568/623

(57) ABSTRACT

The invention relates to highly functional, aliphatic, branched polyethers produced from oxiranes, in particular ethylene oxide and glycidol or the sulfur- or nitrogen-containing analogues thereof. The branched polyethers are in particular suited as a matrix for pharmacological and cosmetic active agents.

The present invention relates to highly functional branched poly(ethylene glycol)s (PEGS) based on substituted and unsubstituted oxiranes, and methods for the production and chemical modification thereof for biomedical, pharmacological and cosmetic applications.

Branched PEG analogs according to the present invention are suitable for the industrial production of biologically and pharmacologically active agents. In addition, the branched PEG analogs can be linked well with non-polar lipid structures, such that they are suitable as selective host molecules for cosmetic or galenic active substances, wherein the release of an active substance, which can be covalently bonded or alternatively non-covalently incorporated, is accurately controllable. Further applications foreseen are coating materials for modifying surfaces or fibers and additives for adhesives or use for the adjustment of the rheological properties of viscous materials.

PEGs or poly(ethylene oxide)s (PEO)s are customarily obtained by anionic ring-opening polymerization of ethylene oxide using a suitable base. On account of its good thermal stability and low reactivity and toxicity, PEG is one of the most frequently used biocompatible water-soluble polymers. Because of its inherent degree of crystallization, the use possibilities of PEG, however, are often restricted.

Further information about PEG and its applications can be gathered from the specialist literature such as, for example, J. M. Harris and S. Zaplinsky, “Poly(ethylene glycol)—Chemistry and Biological Applications”, American Chemical Society, Washington, D.C., 1997.

Hyperbranched polyglycerol (hbPG), similarly to PEG, has outstanding biocompatibility and thermal stability. In contrast to linear PEG, hbPG contains a high number of functional hydroxyl groups, such that various possibilities open up for chemical modification. HbPG is obtained by cationic or anionic ring-opening polymerization of glycidol. The molecular weight and the polydispersity of hbPG are controlled by slow addition, according to the methods described by Sunder et al., “Controlled synthesis of hyperbranched polyglycerols by ring-opening multibranching polymerization” Macromolecules 1999, 32, 4240 or by Wilms et al., “Hyperbranched polyglycerols with elevated molecular weights—a facile two-step synthesis protocol based on polyglycerol macro-initiators” Macromolecules 2009, 42, 3230.

On account of its numerous hydroxyl groups, hbPG is soluble only in water, low molecular weight alcohols and strongly polar aprotic solvents such as pyridine, dimethylformamide (DMF), N-methyl-2-pyrrolidone (NMP) or dimethyl sulfoxide (DMSO), which on account of their high boiling point and/or their toxicity have a restricted application range. In addition, hbPG or the starting product glycidol is expensive and is only suitable for selected applications for economic reasons. Detailed information on hbPG is found in Wilms et al., “Hyperbranched polyglycerols: from the controlled synthesis of biocompatible polyether polyols to multipurpose applications” Acc. Chem. Res. 2009, doi: 10.1021/ar900158p.

For years, great efforts have been made to combine the advantageous properties of PEG and hbPG in one copolymer. On account of the extremely complex polymer chemistry and the very different physical properties of the monomers ethylene oxide and glycidol, only little success was afforded up to now to these efforts.

Thus Hawker et al., “Hyperbranched poly(ethylene glycol)s: a new class of ion-conducting materials” Macromolecules 1996, 29, 3831 describe a multistage method, in which branched PEG derivatives are converted with high preparative outlay to an amorphous material with broad scattering of the molecular weight (polydispersity >2.2) and an interruption of the pure polyether structure by AB₂ macromonomers having aromatic branching points.

Feng et al., “Toward an easy access to dendrimer-like poly(ethylene oxide)s” J. Am. Chem. Soc. 2005, 127, 10956 disclose an iterative method for the production of dendrimer-like PEOs. Highly toxic osmium tetroxide (OsO₄) is employed here to produce polyhydroxylated intermediates. Traces of osmium tetroxide inevitably reach the final product and prevent use for biomedical purposes.

Lapienis et al., “One-pot synthesis of star-shaped macromolecules containing polyglycidol and poly-(ethylene oxide) arms”, Biomacromolecules 2005, 6, 752, and other research groups have thoroughly investigated the synthesis of star-shaped polymers based on PEO. These star-shaped polymers having several—typically 1 to 6—PEG arms, which were described in detail in the course of the last 20 years, can be easily crystallized because of their long linear PEO segments. In contrast, the branch-to-branch structure of branched PEGs, as are described in the present patent application, prevents crystallization to a considerable extent.

However, to date it has not been succeeded in developing a simple and effective method for the production of branched PEGs. The obvious approach, to employ a statistical copolymerization with cyclic glycidol as the latent source for AB₂ branching structures, results in amorphous, biocompatible branched PEG derivatives, which consist exclusively of aliphatic polyether units. However, such a synthesis does not exist up to now. Dimitrov et al., “High molecular weight functionalized poly(ethylene oxide)” Polymer 2002, 43, 7171, report on attempts to copolymerize ethylene oxide and glycidol, wherein a compound with very broad distribution of the molecular weights was obtained. Moreover, a very low incorporation of the glycerol branching units into the polymers is observed. Efforts to control the properties of these copolymers afforded no success up to now.

U.S. Pat. No. 7,196,145 discloses various methods for the statistical copolymerization of ethylene oxide and glycidol. The compounds claimed therein are not in accord with the disclosed data. Obviously, homo-polymerization, which leads to an uncontrolled mixture of homo- and copolymers, occurs to a considerable extent. This is in accord with theoretical considerations and is to be attributed to the use of monofunctional initiator molecules and unsuitable reaction conditions.

By means of the synthesis methods known above, PEG-based compounds are obtained that have one or more of the following disadvantages:
The synthesis is preparatively very laborious, comprises numerous time-intensive steps and/or demanding purification methods and is therefore uneconomical.
The molecular weights of the PEG compounds obtained are not controllable and/or scatter over a wide range and have a high polydispersity.
Use for biological and medical applications is greatly restricted or not justifiable, because the PEG compounds obtained contain no undisturbed polyether segments and/or contain traces of toxic substances such as osmium tetroxide.
The reaction conditions necessary for the synthesis prevent the statistical copolymerization of ethylene oxide with glycidol and promote homo-polymerization.
The PEG compounds obtained contain long PEO segments and virtually without exception crystallize under normal conditions.

The object of the present invention consists in making available a simple and economical method for the production of aliphatic, highly functionalized and (consequently) branched compounds.

This object is achieved by a method comprising the steps:
(a) Mixing of two or more monomers chosen from the group comprising oxiranes, sulfur-containing oxirane analogs and nitrogen-containing oxirane analogs with one or more initiators chosen from the group comprising deprotonated alcohols, amines and amines having protective groups, wherein the initiator(s) have 2 to 100 functional groups and the molar ratio of the monomers to the initiator(s) (I) lies in the range from 6:1 to 5000:1;
(b) Initiation of an anionic ring-opening polymerization; and
(c) Termination of the polymerization by introducing a protic reagent.

Expediently, initiators containing 2 to 50, 3 to 50, 4 to 30, 4 to 20 or 10 to 15 functional groups are used in step (a).

Preferably, the oxiranes ethylene oxide (1,2-epoxy-ethane) and glycidol (oxiranylmethanol) with molar fractions of 0.5 to 97% or of 3 to 99%, based on the totality of the monomers, are employed in step (a).

In particular, at least one monomer chosen from the group comprising propylene oxide (1,2-epoxypropane), 1,2-epoxybutane (ethyloxirane), allyl glycidyl ether (1-allyloxy-2,3-epoxypropane), benzyl glycidyl ether (benzyloxymethyloxirane), tert-butyl glycidyl ether (tert-butoxymethyloxirane), ethoxyethyl glycidyl ether, styrene oxide (2-phenyloxirane), aziridine (ethylene-imine) and thiirane (ethylene sulfide) is employed in step (a), wherein the molar fraction of the at least one monomer chosen from the group is 1 to 30%, based on the totality of the monomers.

Further embodiments of the method according to the invention are distinguished in that:
in step (a) an alcohol having 2 to 100, 2 to 50, 3 to 50, 4 to 30, 4 to 20 or 10 to 15 functional groups is employed as initiator, wherein 5 to 30% of the functional groups are deprotonated;
in step (a) an amine is employed as initiator;
in step (a) an agent chosen from the group comprising alkali metal naphthalides, diphenylmethyl alkali metals, alkali metals, hydroxides, hydrides, alkoxides and mixtures thereof is employed for deprotonating the initiator;
volatile byproducts of the deprotonation are removed from the reaction mixture;
the method is carried out in a low-pressure atmosphere of 0.0001 to 0.95 bar;
the method is carried out in a high-pressure atmosphere of 1.1 to 40 bar;
the method is carried out at a temperature of 40 to 150° C.;
in step (a) one or more of the monomers is (are) introduced continuously;
in step (a) a sulfur-containing oxirane analog is employed as one of the monomers;
in step (c) an alcohol or water is used to terminate the polymerization; and
the method comprises a further step (d), in which the compound obtained in step (c) is reacted with a functionalizing reagent, wherein the reagent reacts with OH groups of said compound.

The method according to the invention makes possible the production of highly functionalized branched PEG derivatives (in the following designated as bPEG), wherein the properties of the bPEGs obtained, such as functionalization, thermal and chemical stability, biocompatibility as well as viscosity and solubility, are suited in a targeted manner to specific applications.

The use of polyfunctional initiators having at least 2 functional groups is an important prerequisite for obtaining bPEG having a low polydispersity M̅_{w}/ M̅ₙ<3.

In particular, it was found that bPEG with desired properties are obtained by copolymerizing various substituted or non-substituted oxiranes with glycidol in a determined molar ratio variable within wide ranges.

Accordingly, the invention creates a method for the production of highly functionalized aliphatic bPEGs, which in an advantageous embodiment comprises at least the following steps:
(i) Providing an initiator core by 1) deprotonation of a functionalized alcohol having at least 2 hydroxyl groups or 2) addition of an amine which optionally contains protective groups;
(ii) Initiation of a polymerization by reacting ethylene oxide and glycidol and also optionally further substituted oxiranes with the initiator core in a prespecified molar ratio;
(iii) Termination of the polymerization; and
(iv) Isolation of the copolymer.

A further object of the invention consists in creating highly functionalized branched compounds having properties that are adjustable, i.e. suited for a certain application. This object is achieved by a functionalized branched compound, comprising:
an initiator core consisting of an initiator having 2 to 100 functional groups chosen from the group comprising deprotonated alcohols, amines and amines having protective groups;
linear polyether segments (K) consisting of monomer units (M); and
branching sites (D),
   wherein the branching sites (D) and the monomer units (M) are formed from monomers chosen from the group comprising oxiranes, sulfur-containing oxirane analogs and nitrogen-containing oxirane analogs, and the molar ratio of branching sites (D) and monomer units (M) to the initiator core (I) lies in the range from 6:1 to 5000:1.

In particular, the functionalized branched compound comprises an initiator core consisting of an initiator having 2 to 50, 3 to 50, 4 to 30, 4 to 20 or 10 to 15 functional groups.

Preferably, the branching sites (D) and monomer units (M) are formed from the oxiranes ethylene oxide and glycidol, wherein the molar fractions of ethylene oxide are 0.5 to 97% and of glycidol 3 to 99.5%, based on the totality of the branching sites (D) and monomer units (M).

In particular, the branching sites (D) and monomer units (M) are formed from at least one monomer chosen from the group comprising propylene oxide (1,2-epoxypropane), 1,2-epoxybutane (ethyloxirane), allyl glycidyl ether (1-allyloxy-2,3-epoxypropane), benzyl glycidyl ether (benzyloxymethyloxirane), tert-butyl glycidyl ether (tert-butoxymethyloxirane), ethoxyethyl glycidyl ether, styrene oxide (2-phenyloxirane), aziridine (ethyleneimine) and thiirane (ethylene sulfide).

Further embodiments of the compound according to the invention are characterized in that:
it contains 5 to 1000 functional OH groups; it has a molecular weight of 400 to 100000 g/mol, preferably of 10000 to 30000 g/mol;
it has a polydispersity M̅_{w}/ M̅ₙ of 1 to 10, preferably of 1 to 3 and in particular 1.3 to 2.3;
the ratio (N_{D}/N_{M}) of the number (N_{D}) of branching sites (D) to the number (N_{M}) of the monomer units (M) lies in the range from 1:100 to 80:100, preferably 10:100 to 60:100 and in particular 10:100 to 20:100; and
at least two dendrites are bonded to the initiator core, wherein the dendrites consist of linear polyether segments (K) of monomer units (M) and branching sites (D).

The compounds according to the invention are in particular macromolecular, non-crosslinked, non-linear bPEGs that comprise numerous hydroxyl groups or other end groups with specific electrophilic subgroups and a likewise functionalized initiator core.

The invention makes available bPEG materials, whose properties, such as general chemical composition, molecular weight, polydispersity, chemical composition and functionalization of the initiator core, total number of functional groups, degree of branching and chemical composition of the end groups, are selectively adjustable in a wide range.

The bPEGs according to the invention are preferably produced by a method as claimed in claims 1 to 15.

In the following, the invention creates a method for the modification of highly functionalized branched compounds such as bPEG by esterification, etherification, urethane formation and silylation.

The invention is illustrated in more detail below by means of drawings and examples. The drawings show:

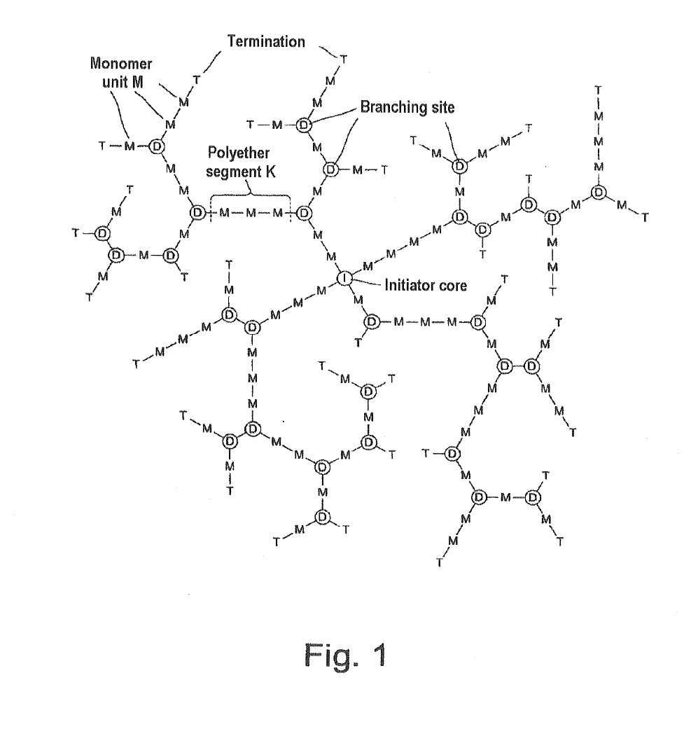

FIG. 1 a schematic representation of the structure of the compounds according to the invention;

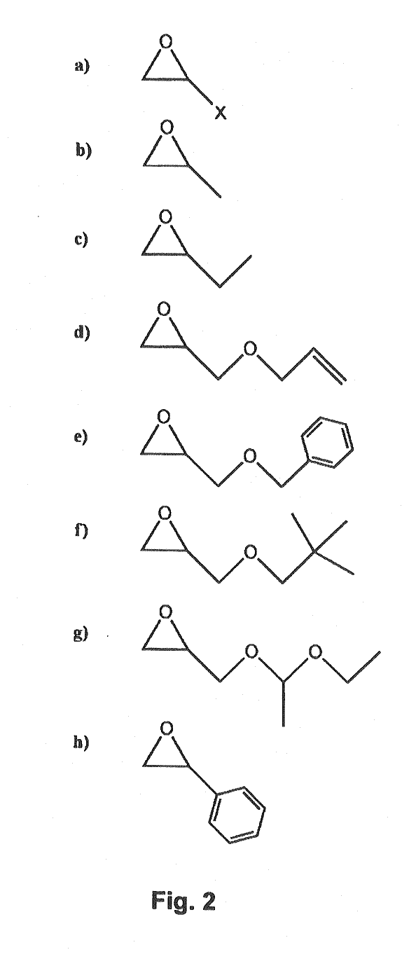

FIG. 2 examples of oxiranes that are used for the production of the compounds according to the invention;

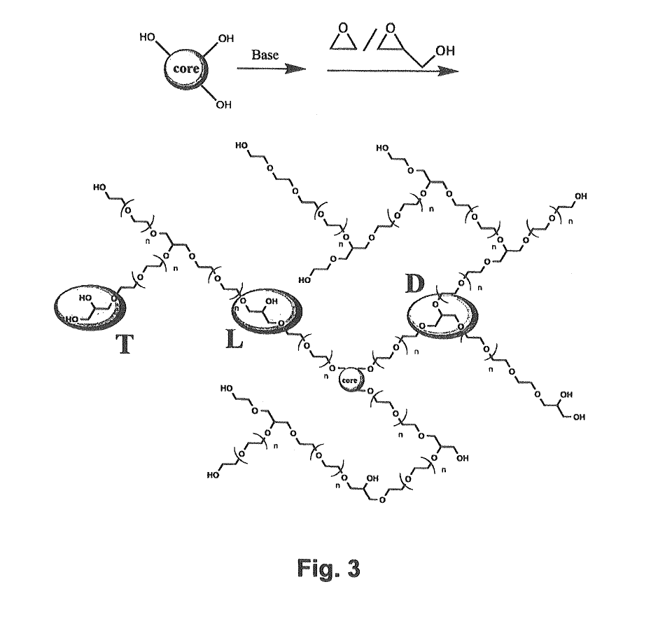

FIG. 3 the structure of a bPEG produced by means of the oxiranes ethylene oxide and glycidol;

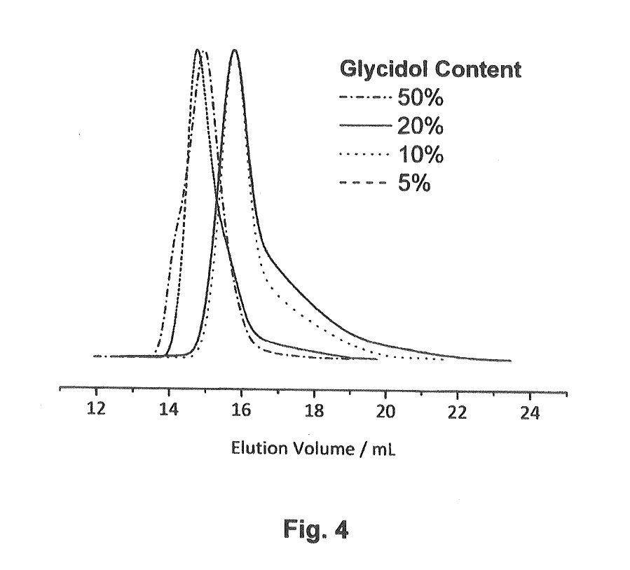

FIG. 4 elution profiles of bPEGs as a function of the glycidol content; and

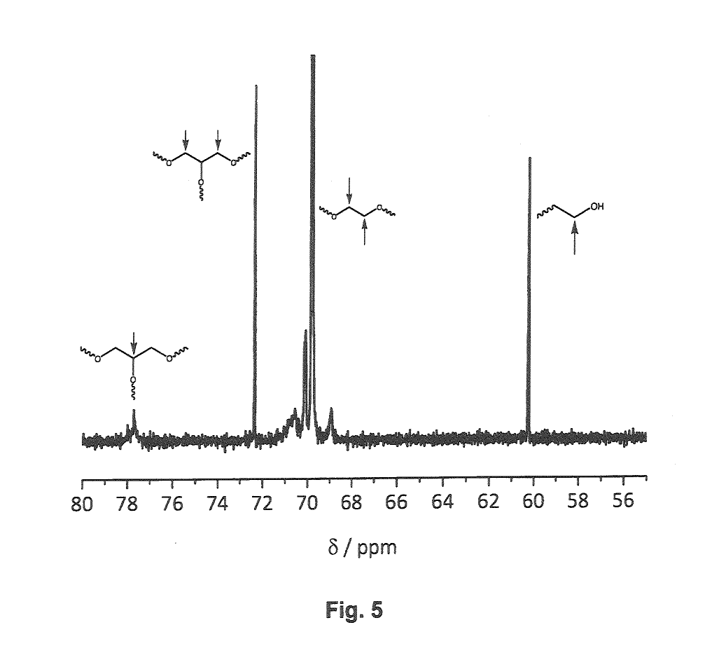

FIG. 5 an NMR spectrum of a bPEG according to the invention.

FIG. 1 shows a schematic representation of a compound according to the invention having an initiator core I, linear polyether segments K consisting of monomer units M and branching sites D. The polyether segments K and also some of the branching sites D have terminal groups T. 2 to 100, 2 to 50, 3 to 50, 4 to 30, 4 to 20 or 10 to 15 dendrites can be bound to the initiator core, wherein each dendrite consists of linear polyether segments K consisting of monomer units M and branching sites D and has a more or less branched structure.

As an initiator, all sorts of alcohols or amines having 2 to 100, 2 to 50, 3 to 50, 4 to 30, 4 to 20 or 10 to 15 functional hydroxyl or amino groups are suitable for the production of the branched compounds according to the invention. For example, a suitable initiator is substances such as pentaerythritol, sorbitol, glycerol, trimethylolpropane, di(benzyl)aminoalcohols, commercial polyglycerols such as Diglycerol®, Polyglycerol-3® and Polyglycerol-4®, commercial polyether polyols such as Arcol®, Desmpophen®, Hyperlite®, Baygal® or Ultracell® as well as a multiplicity of amines with and without protective groups.

The deprotonation of the alcohols employed as an initiator takes place by means of strong bases. Suitable bases are alkali metals, in particular sodium, potassium and cesium; hydroxides such as sodium, potassium and cesium hydroxide; alkali metal hydrides, such as potassium hydride, alkoxides such as sodium methylate, potassium methylate, sodium ethoxide or potassium tert-butylate, sodium naphthalide, potassium naphthalide, diphenylmethyl sodium or diphenylmethyl potassium.

The polymerization is carried out by reacting the initiator—if appropriate after deprotonation—with at least two monomers. According to the invention, the at least two monomers are chosen from the group comprising substituted and non-substituted oxiranes and sulfur- or nitrogen-containing oxirane analogs. Preferably, the oxiranes ethylene oxide and glycidol are used. Optionally a further substituted oxirane is employed which has a group X (see FIG. 2a) that imparts further orthogonal functionalities to the compound and/or modifies its solubility and thermal properties. FIG. 2 shows examples of oxiranes that are employed according to the invention, wherein the letters (b) to (h) designate the following oxiranes:

(b) propylene oxide (1,2-epoxypropane),
(c) 1,2-epoxybutane (ethyloxirane),
(d) allyl glycidyl ether (1-allyloxy-2,3-epoxy-propane),
(e) benzyl glycidyl ether (benzyloxymethyloxirane),
(f) tert-butyl glycidyl ether (tert-butoxymethyl-oxirane),
(g) ethoxyethyl glycidyl ether, and
(h) styrene oxide (2-phenyloxirane).

In addition to the above-substituted oxiranes, sulfur- or nitrogen-containing oxirane analogs such as aziridine (ethyleneimine) and thiirane (ethylene sulfide) are suitable.

The production and general structure of a bPEG according to the invention are shown in FIG. 3. The bPEG molecule contains an initiator core (“core”) and ethylene glycol and glycerol units. The various structural units are emphasized and designated by the letters “D” for a branched glycerol unit, “L” for a linear or unbranched glycerol unit and “T” for a terminating glycerol unit.

If, as in the case of the compound shown in FIG. 3, glycidol is used in production, on opening the C₂H₄O rings and on incorporation of the glycerol unit secondary alkoxides are formed, which can change into primary alkoxides by means of intra- or intermolecular proton transfer. If both alkoxides of the glycerol unit propagate in the course of the polymerization, a branching site D results. In the case that only one alkoxide of a glycerol unit reacts with another monomer such as ethylene oxide, a linear unit L is formed. Terminating units T result if neither of the two alkoxides of a glycerol unit has reacted on ending the polymerization by addition of a protic reagent such as methanol or water.

In the case that an alcohol is employed as an initiator, this is deprotonated by protons of the alcohol group being removed. The degree of deprotonation of the hydroxyl groups is varied from 5 to 100%. If a protic compound such as, for example, methanol is formed in the deprotonation when using methylates as a base, this can be removed by applying a reduced pressure before the start of the polymerization.

The composition of the copolymers or polymers is adjusted by adjusting the ratio of the at least two monomers. For example, ethylene oxide is added in a prespecified proportion of between 0.5 and 97 mol %, based on the totality of monomers. The higher the content of ethylene oxide relative to glycidol and optionally a further monomer, the longer linear PEG chains the polymer contains. Preferably, ethylene oxide is added to the reaction vessel, which contains an initiator dried by means of distillation and condensed in vacuo. Glycidol and optionally one or more of the above-mentioned monomers, in particular those shown in FIGS. 2 b) to h), are added before or after the ethylene oxide.

The molecular weight of the polymers is adjusted by the molar ratio of the monomers relative to the initiator. By way of approximation, the number of the functional hydroxyl groups n(OH) per polymer molecule depends on the content of the incorporated glycidol units and the functionalization of the initiator core, because both the glycidol units and the initiator core contribute hydroxyl groups. This approximation applies only if the homopolymerization is negligible without the admission of the initiator core. According to the invention, the number of hydroxyl groups n(OH) per polymer molecule is between 5 and 1000. To end the polymerization and to obtain the desired bPEG, a protic reagent is introduced.

The size of the polymers produced according to the invention is analyzed by means of gel permeation chromatography (GPC). It is seen here that the content of low molecular weight polymers is less than 5 mol %. This low molecular weight content can easily be removed by means of precipitation or dialysis.

FIG. 5 shows the GPC elugrams obtained from four polymers produced according to the invention containing different contents of ethylene glycol and glycidol units after separation of the low molecular weight polymers by means of size exclusion chromatography.

The molecular weights of the bPEGs obtained preferably lie in a range between 400 and 100000 g/mol. The scattering of the molecular weights (polydispersity M̅_{w}/ M̅ₙ) is low and lies in a range from 1 to 3 and in particular from 1.3 to 2.3; occasionally larger values of up to 10 are observed.

The statistical branching structure of the polymers obtained is analyzed by means of nuclear magnetic resonance spectroscopy (NMR). In the case of poly(ethylene oxide)-co-(glycidol), the hydrogen atoms can be differentiated according to their chemical bonding by means of ¹H-NMR spectroscopy. The quantitative evaluation of the ¹H-NMR spectra allows the relative number of different hydrogen-containing groups to be determined. For this, the intensities or areas of the resonance lines (or resonance peaks) occurring at characteristic frequencies (or frequency shifts) are integrated and compared with one another. Customarily, the integration is carried out numerically supported by software, if appropriate with the aid of a model function (non-linear least squares fit) adapted by nonlinear regression to the resonance line measured. By means of the peak intensities of the ¹H-NMR spectra, different ratios as well as the molecular weight can be determined: (i) number of hydrogen atoms present in OH groups of the glycidol units relative to the number of the hydrogen present in the initiator core; (ii) number of hydrogen atoms in ethylene oxide units, by subtracting the hydrogen signal of the glycidol units from the signal of the repeating units; and (iii) addition of the molecular mass of the initiator core and the corresponding monomer units. The molecular weights determined by means of method (iii) agree well with the values obtained by GPC.

In addition, the molecular structure of the polymers according to the invention was determined by means of “inverse gated” ¹³C-NMR spectroscopy. ¹³C-NMR spectroscopy allows details of the monomer units present in a molecule to be determined. In FIG. 5, a section of a ¹³C-NMR spectrum measured on a bPEG according to the invention is shown. The symbols shown next to the resonance lines indicate from which carbon atom or from which structural unit the resonance signal results. By integration of the various resonance lines, the number of monomer units per molecule can be determined.

The characteristic parameters of some bPEGs according to the invention, comprising an initiator core, ethylene glycol and glycidol units, are shown in Table 1.

TABLE 1

| Glycidol | Glycidol | Glycidol | Molecular weight | Polydispersity |
|---|---|---|---|---|
| % | % | % | g/mol | — |
| (calc.*) | (¹H NMR) | (¹³C NMR) | (SEC) | (SEC) |
| 50 | 45.1 | 37.1 | 38 000 | 1.34 |
| 20 | 14.5 | 13.8 | 16 000 | 1.81 |
| 15 | 12.2 | 11.3 | 29 000 | 1.76 |
| 12 | 11.4 | 9.5 | 27 000 | 1.38 |
| 10 | 8.8 | 8.7 | 39 000 | 1.36 |
| 5 | 6.6 | 4.6 | 27 000 | 1.34 |
| 3 | 3.9 | 3.8 | 17 000 | 1.41 |

*calculated by means of the amount of glycidol employed for the reaction

The solubility of the bPEGs according to the invention is essentially determined by the ratio of the comonomers. All bPEGs are soluble in water, low molecular weight alcohols and strongly polar aprotic solvents such as dimethylformamide (DMF) or dimethyl sulfoxide (DMSO). The greater the relative content of ethylene glycol units, the better the solubility in hydrophobic solvents such as chloroform. All polymers with a PEG content of greater than 5 mol % do not crystallize at room temperature. The polymers are present in various forms at room temperature, for example as a clear viscous liquid or as a waxy substance.

The thermal properties of the highly functionalized bPEGs according to the invention can be adjusted by means of the relative proportions of the copolymers. The glass transition temperature of the bPEGs as a rule lies below −50° C. The melt enthalpy of the bPEGs is a function of the PEG content.

TABLE 2

| Glycidol | T_{g} | Tₘ | Melt enthalpy |
|---|---|---|---|
| % (calculated*) | ° C.** | ° C.** | J/g** |
| 50 | −60.1 | — | — |
| 20 | −62.0 | −20.8 | 9.2 |
| 12 | −61.0 | 4.8 | 35.2 |
| 10 | −58.7 | 6.7 | 49.3 |
| 5 | −57.7 | 18.4 | 69.8 |
| 3 | −60.0 | 33.3 | 102.1 |

*calculated by means of the amount of glycidol employed for the reaction
**determined by means of differential scanning calorimetry (DSC)

In addition, the invention relates to methods for the modification of the functional branched compounds as claimed in claims 16 to 23 by esterification or silylation. These modifications serve to increase the solubility in a polar solvents and/or to adjust the viscosity. Depending on functionalization, various uses of the compounds according to the invention are foreseen.

Thus the compounds according to the invention are suitable for the production of sunscreen compositions. For this, the functional branched compounds are linked with UV absorbers. Preferably, the functional branched compounds are used for the production of medicaments, cosmetics, shampoos, lotions and hygiene articles. Moreover, the use in technical application fields such as the production of lubricants, adhesives and synthetic fibers is provided.

Exemplary synthesis methods for the production of the compounds according to the invention are shown below.

### EXAMPLE 1

### Production of bPEG with 3% Glycidol and Trimethylol-Propane as Initiator

A glass reaction vessel having two nozzles, a septum, a Teflon tap and a magnetic stirrer is connected to a vacuum pump and evacuated. 0.134 g (1 mmol) of trimethylolpropane is added to the reaction vessel and suspended in 5 ml of benzene. After stirring the suspension for 30 minutes, we kept the reaction vessel in vacuo fork at least 3 hours to remove all traces of water and other readily volatile substances azeotropically. The reaction vessel is then filled with argon, 30 ml of freshly distilled diethylene glycol dimethyl ether (diglyme) is added to the trimethylol-propane to dissolve trimethylolpropane and freshly prepared potassium naphthalide is added dropwise in the form of 1.03 ml of a 0.29 M tetrahydrofuran solution with continuous stirring. Subsequently, the initiator solution is stirred at room temperature for 1 h. The initiator solution obtained is cooled to a temperature of −80° C. and the reaction vessel is evacuated. 7 ml of ethylene oxide, corresponding to 140 mmol, are dried over calcium hydride in an ampoule and transferred to the reaction vessel in vacuo. The reaction vessel is sealed and 0.3 ml, corresponding to 4.5 mmol, of freshly distilled glycidol are added through the septum by means of a syringe. The reaction mixture is heated to a temperature of 80° C. and stirred for a period of 18 h. After addition of an excess amount of methanol, the reaction mixture is dialyzed against methanol or water for a period of 3 days. Finally, methanol or water is removed in vacuo at a temperature of 60° C. over a period of 24 h to obtain the polymer according to the invention with a yield of 80 to 90% of the reaction mixture.

### EXAMPLE 2

### Production of bPEG Using 3% Glycidol and N,N-di-(benzyl)amino-1,3-propanediol as Initiator

A glass reaction vessel having two nozzles, a septum, a Teflon tap and a magnetic stirrer is connected to a vacuum pump and evacuated. 0.271 g (1 mmol) of N,N-di(benzyl)amino-1,3-propanediol is transferred to the reaction vessel and dissolved in 5 ml of anhydrous benzene. The reaction vessel is evacuated and kept in vacuo for at least 3 hours to remove all traces of water and other readily volatile substances. The reaction vessel is then filled with argon, 30 ml of freshly distilled diethylene glycol dimethyl ether (diglyme) are added to the initiator to dissolve the N,N-di(benzyl)amino-1,3-propanediol and freshly prepared potassium naphthalide is added dropwise with continuous stirring in the form of 0.7 ml of a 0.29 M tetrahydrofuran solution. Subsequently, the initiator solution is stirred at room temperature for 1 h. The initiator solution obtained is cooled to a temperature of −80° C. and the reaction vessel is evacuated. 7 ml of ethylene oxide, corresponding to 140 mmol, are dried over calcium hydride in an ampoule and transferred to the reaction vessel in vacuo. The reaction vessel is sealed and 0.3 ml, corresponding to 4.5 mmol, of freshly distilled glycidol are added through the septum by means of a syringe. The reaction mixture is heated to a temperature of 80° C. and stirred for a period of 18 h. After addition of an excess amount of methanol, the solution is neutralized using a cation exchange resin (Dowex 50WX8) and stirred at room temperature for one hour. All solvents are removed under low pressure and the reaction mixture is dissolved in 10 ml of methanol. After precipitation in cold diethyl ether and drying in vacuo at a temperature of 60° C. for a period of 24 h, a waxy polymer is obtained in a yield of 80 to 90% of the reaction mixture.

### EXAMPLE 3

### Production of bPEG Using 10% Glycidol and N,N-di-(benzyl)amino-1,3-propanediol as Initiator

A glass reaction vessel having two nozzles, a septum, a Teflon tap and a magnetic stirrer is connected to a vacuum pump and evacuated. 0.271 g (1 mmol) of N,N-di(benzyl)amino-1,3-propanediol is transferred to the reaction vessel and dissolved in 5 ml of anhydrous benzene. The reaction vessel is evacuated and kept in vacuo for at least 3 hours to remove all traces of water and other readily volatile substances. The reaction vessel is then filled with argon, 30 ml of freshly distilled diethylene glycol dimethyl ether (diglyme) are added to the initiator to dissolve the N,N-di(benzyl)amino-1,3-propanediol, and freshly prepared potassium naphthalide is added dropwise with continuous stirring in the form of 0.7 ml of a 0.29 M tetrahydrofuran solution. Subsequently, the initiator solution is stirred at room temperature for 1 h. The initiator solution obtained is cooled to a temperature of −80° C. and the reaction vessel is evacuated. 7 ml of ethylene oxide, corresponding to 140 mmol, are dried over calcium hydride in an ampoule and transferred to the reaction vessel in vacuo. The reaction vessel is sealed and 1 ml, corresponding to 15 mmol, of freshly distilled glycidol are added through the septum by means of a syringe. The reaction mixture is heated to a temperature of 80° C. and stirred for a period of 18 h. After addition of an excess amount of methanol, the solution is neutralized with a cation exchange resin (Dowex 50WX8) and stirred at room temperature for one hour. All solvents are removed under low pressure and the reaction mixture is dissolved in 10 ml of methanol. After precipitation in cold diethyl ether and drying in vacuo at a temperature of 60° C. for a period of 24 h, a clear and viscous polymer is obtained in a yield of 80 to 90% of the reaction mixture.

1. A method for the production of functional branched compounds comprising the steps:
(a) mixing two or more monomers chosen from the group comprising oxiranes, sulfur-containing oxirane analogs and nitrogen-containing oxirane analogs with one or more initiators chosen from the group comprising deprotonated alcohols, amines and amines having protective groups,
wherein the initiator(s) have 2 to 100 functional groups and the molar ratio of the monomers to the initiator(s) lies in the range from 6:1 to 5000:1;
(b) initiating an anionic ring-opening polymerization; and
(c) terminating the polymerization by introducing a protic reagent.

2. The method as claimed in claim 1, wherein in step (a) the oxiranes are ethylene oxide and glycidol with molar fractions of 0.5 to 97% or of 3 to 99%, based on the totality of the monomers.

3. The method as claimed in claim 1, wherein in step (a) at least one monomer is chosen from the group comprising propylene oxide (1,2-epoxypropane), 1,2-epoxybutane (ethyloxirane), allyl glycidyl ether (1-allyloxy-2,3-epoxypropane), benzyl glycidyl ether (benzyloxymethyloxirane), tert-butyl glycidyl ether (tert-butoxymethyloxirane), ethoxyethyl glycidyl ether, styrene oxide (2-phenyloxirane), aziridine (ethyleneimine) and thiirane (ethylene sulfide), wherein the molar fraction of the at least one monomer chosen from the group is 1 to 30%, based on the totality of the monomers.

4. The method as claimed in claim 1, wherein step (a) includes an alcohol having 2 to 100 functional groups as initiator and 5 to 30% of the functional groups are deprotonated.

5. The method as claimed in claim 1, wherein step (a) includes an amine as initiator.

6. The method as claimed in claim 1, wherein step (a) further includes an agent chosen from the group comprising alkali metal naphthalides, diphenylmethyl alkali metals, alkali metals, hydroxides, alkoxides and mixtures thereof to deprotonate the initiator.

7. The method as claimed in claim 6, wherein volatile byproducts of the deprotonation are removed from the reaction mixture.

8. The method as claimed in claim 1, wherein said method is carried out in a low-pressure atmosphere of 0.0001 to 0.95 bar.

9. The method as claimed in claim 1, wherein said method is carried out in a high-pressure atmosphere of 1.1 to 40 bar.

10. The method as claimed in claim 1, wherein said method is carried out at a temperature of 40 to 150° C.

11. The method as claimed in claim 1, wherein step (a) further comprises continuously introducing one or more of the monomers.

12. The method as claimed in claim 1, wherein in step (a) one of the monomers is a sulfur-containing oxirane analog.

13. The method as claimed in claim 1, wherein in step (c) an alcohol or water is introduced to terminate the polymerization.

14. The method as claimed in claim 1, wherein said method comprises a further step (d) comprising reacting the compound obtained in step (c) with a functionalizing reagent, wherein the reagent reacts with OH groups of said compound.

15. The method as claimed in claim 1, wherein step (c) further comprises adding compounds having functional groups different from OH to the reaction mixture.

16. A functional branched compound comprising
an initiator core consisting of an initiator having 2 to 100 functional groups chosen from the group comprising deprotonated alcohols, amines and amines having protective groups;
linear polyether segments consisting of monomer units; and
branching sites;
wherein the branching sites and the monomer units are formed from monomers chosen from the group comprising oxiranes, sulfur-containing oxirane analogs and nitrogen-containing oxirane analogs, and the molar ratio of branching sites and monomer units to the initiator core lies in the range from 6:1 to 5000:1.

17. The compound as claimed in claim 16, wherein said compound comprises branching sites and monomer units of the oxiranes ethylene oxide and glycidol having molar fractions of 0.5 to 97% or 3 to 99.5%, based on the totality of the branching sites and monomer units.

18. The compound as claimed in claim 16, wherein said compound comprises branching sites and monomer units of at least one monomer chosen from the group comprising propylene oxide (1,2-epoxypropane), epoxybutane (ethyloxirane), allyl glycidyl ether (1-allyloxy-2,3-epoxypropane), benzyl glycidyl ether (benzyloxymethyloxirane), tert-butyl glycidyl ether (tert-butoxymethyloxirane), ethoxyethyl glycidyl ether, styrene oxide (2-phenyloxirane), aziridine (ethyleneimine) and thiirane (ethylene sulfide).

19. The compound as claimed in claim 16, wherein said compound contains 5 to 1000 functional OH groups.

20. The compound as claimed in claim 16, wherein said compound has a molecular weight of 400 to 100000 g/mol.

21. The compound as claimed in claim 16, wherein said compound has a polydispersity M̅_{w}/ M̅ₙ of 1 to 10.

22. The compound as claimed in claim 16, wherein the ratio (N_{D}/N_{M}) of the number (N_{D}) of branching sites to the number (N_{M}) of the monomer units lies in the range from 1:100 to 80:100.

23. The compound as claimed in claim 16, wherein said compound is formed by a method as claimed in claim 1.

24. A sunscreen composition comprising a functional branched compound as claimed in claim 16 and at least one UV absorber.

25. Cosmetics, shampoos, lotions and hygiene articles comprising a functional branched compound as claimed in claim 16.

26. Medicaments comprising a functional branched compound as claimed in claim 16.

27. Lubricants, adhesives and synthetic fibers comprising a functional branched compound as claimed in claim 16.

28. The method as claimed in claim 15, wherein step (c) comprises adding further compounds having amino, carboxylic acid, carbonyl, sulfonic acid and aldehyde groups to the reaction mixture.

29. The compound as claimed in claim 20, wherein said compound has a molecular weight of 10000 to 30000 g/mol.

30. The compound as claimed in claim 21, wherein said compound has a polydispersity M̅_{w}/ M̅ₙ of 1 to 3.

31. The compound as claimed in claim 21, wherein said compound has a polydispersity M̅_{w}/ M̅ₙ of 1.3 to 2.3.

32. The compound as claimed in claim 22, wherein the ratio of the number of branching sites to the number of the monomer units lies in the range from 10:100 to 60:100.

33. The compound as claimed in claim 22, wherein the ratio of the number of branching sites to the number of the monomer units lies in the range from 10:100 to 20:100.
